(19) United States
(12) Patent Application Publication
(10) Pub. No.: US 2020/0124619 A1
(43) Pub. Date: Apr. 23, 2020

(54) BIOMARKERS FOR LIVER FUNCTION

(71) Applicant: PharmK Corporation, Jamison, PA (US)

(72) Inventor: Yuhong He, Dublin, CA (US)

(73) Assignee: PharmK Corporation, Jamison, PA (US)

(21) Appl. No.: 16/626,776

(22) PCT Filed: Oct. 25, 2018
(86) PCT No.: PCT/US18/57615

§ 371 Date: Dec. 26, 2019

Related U.S. Application Data
(60) Provisional application No. 62/577,160, filed on Oct. 25, 2017.

(51) Int. Cl. G01N 33/68

(52) U.S. Cl. CPC G01N 33/6893; G01N 2800/56; G01N 2800/52; G01N 2800/085

(57) ABSTRACT

The present invention provides convenient, noninvasive methods for the sensitive and early stage diagnosis of liver functions. The methods comprise measuring coproporphyrin I and II levels in, for example, plasma, as an indicator of functional changes in the liver, that may include, but are not limited to changes due to drug-related inhibition of hepatic transport, acute or chronic liver diseases, liver fibrosis or hepatocyte damage.

### RELATED APPLICATIONS

This application is a National Stage application under 35 U.S.C. § 371 of PCT/US2018/057615, filed on Oct. 25, 2018, which claims the benefit of priority under 35 U.S.C. § 119 of U.S. Provisional Application Ser. No. 62/577,160, filed on Oct. 25, 2017, the entire disclosures of which are incorporated herein by reference.

### FIELD OF THE INVENTION

The present invention relates generally to biomarkers. More particularly, the invention relates to biomarkers for early stage liver disease that can be detected non-invasively.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### BACKGROUND

In hepatology, biomarkers are a growing area of research. This research is driven by a global burden of liver disease, the absence of symptoms until late in the natural progression of a disease that may take years to manifest, the lack of a non-invasive reference test (to replace an invasive liver biopsy) to assess the severity of a disease, and a lack of tools to determine the efficacy of therapeutic interventions.

As discussed in Aithal, et al. “Biomarkers in Liver Disease: Emerging Methods and Potential Applications,” (Int. J. Hepatology, Vol. 2012, Article ID 437508, 4 pages, doi: 10.1155/2012/437508), the National Institute of Health defines a biomarker as, a “characteristic that is objectively measured and evaluated as an indicator of normal biologic processes, pathogenic processes, or pharmacologic responses to a therapeutic intervention”. Moreover, biomarkers can be classified into hierarchical systems based on their ability to assess natural history (type 0: prognosis), biological activity (type 1: response to therapy), and therapeutic efficacy (type 2: surrogate for clinical efficacy).”

Certain diseases can result in damage to the liver or impaired liver function. In addition, taking certain drugs or exposure to toxins or heaving metals can also result in damage to the liver or impaired liver function. Acute and chronic hepatitis, liver fibrosis, nonalcoholic fatty liver disease, nonalcoholic steatohepatitis and liver tumor are often associated with liver damage and improper or insufficient liver function. Intake of many drugs can also cause drug-induced liver injury or reduction of hepatic function as a result of drug-drug interactions.

### Symptoms of Liver Disease

Signs and symptoms of liver disease include nausea, vomiting, body aches and malaise that may be mistaken for other illnesses, such as the flu. Additional and more specific symptoms include pruritus, dark urine, pale stools, jaundice, ascites, encephalopathy, portal hypertension and variceal bleeding. Unfortunately, many of these symptoms do not present until disease and damage to the liver is advanced and treatments options are vanishingly more limited. Thus, there has been a continual focus on identifying biomarkers that correlate specifically to liver damage and disease before these symptoms appear.

### Assessment of Liver Function and Diagnosis of Liver Disease

Currently, to determine whether a subject's liver is functioning properly, a medical professional will perform a blood test, an imaging test, and/or or obtain a biopsy from the subject person.

Routine blood tests for liver function measure levels of liver enzymes, such as alanine aminotransferase (ALT) and aspartate aminotransferase (AST). The increased level of these liver enzymes indicates liver damage and correlates to certain liver diseases. However, chronic liver diseases, such as nonalcoholic fatty liver disease and viral infections, are usually asymptomatic during the early stage of the disease or infection. In addition, diseases such as hepatic necroinflammation and fibrosis are often undetectable with blood tests for ALT and AST, as these diseases often show normal levels of ALT and AST.

Since the liver is an organ that produces many proteins, including albumin, decreased albumin levels in plasma can also be used as an indicator of impaired liver function. However, decreased levels of albumin are often only seen during the later stages of liver disease. Thus, measuring albumin levels in plasma is not a sensitive marker for the early diagnose of impaired liver function.

Other blood tests that may be used to asses liver function include: bilirubin (total, conjugated and unconjugated), a breakdown product of heme that is normally cleared by the liver; alkaline phosphatase, which is found in cells lining the biliary ducts of the liver which can be elevated in liver cirrhosis and hepatitis, particularly acute viral hepatitis; gamma glutamyl transpeptidase, an enzyme found in hepatocytes and biliary epithelial cells, as well as other tissues that is helps in glutathione metabolism by transporting peptides across the cell membrane and can be elevated in patients with viral hepatitis, alcoholic and non-alcoholic liver disease.

Other tests may also be ordered to rule out specific causes, including: 5′ nucleotidase, a glycoprotein found throughout the body, that can be increased in raised in obstructive jaundice, parenchymal liver disease, and liver metastases; ceruloplasmin is an acute phase protein synthesized in the liver that is elevated in non-Wilson liver disease and obstructive jaundice; alpha-fetoprotein, a fetal protein similar to albumin that is synthesized in the liver and is an indicator of hepatocellular carcinoma as well as liver injury; the liver is responsible for the production of the vast majority of coagulation factors and impaired blood clotting (determined by prothrombin time and its derived measures of prothrombin ratio and international normalized ratio) can be an indicator of liver disease; serum glucose is related to measures the liver's ability to produce glucose and is usually the last function to be lost in fulminant liver failure; lactate dehydrogenase, which is found in many tissues including liver and may be elevated in liver damage.

Imaging tests for the liver, including computerized tomography scans and magnetic resonance imaging tests require an injection of a special dye or contrast medium into a person. For example, liver function can be based on the measurement of liver-specific contrast agents, such as Gd-EOB-DTPA (Primovist®, Bayer Schering Pharma AG, Berlin, Germany) and Gd-BOPTA (Multihance®, Bracco Imaging, Milan, Italy). Thus, these imaging tests, are considered invasive methods to assess functional changes in the liver. Less invasive imaging by ultrasonography can provide only limited information on the status of the liver, such as size, shape and involvement of the hepatic portal and bile ducts in liver disease and damage.

A great amount of information can be obtained from the most invasive testing: liver biopsy. However, small, focal lesions may be missed with the typical blind biopsy, and liver disease is typically quite advanced at the time a biopsy will provide diagnostic evidence of disease.

The current methods to determine if a person's liver is functioning properly are inconvenient, invasive, and often expensive. Moreover, many of the less invasive blood test are non-specific for liver disease or injury or for a particular pathology. In addition, none of the current methods can be used for continuous or semi-continuous monitoring of temporal or momentary liver functional changes, such as drug-related inhibition of hepatic clearance. In other words, there currently are no methods to monitor liver function after treatment with a medication to determine whether recovery of the liver, i.e. a return to normal liver function, is progressing satisfactorily.

There is a need for a noninvasive, highly sensitive, and convenient biomarker-based test to determine whether a person's liver is functioning properly that can be used early in the course of disease progression, to monitor efficacy of treatment and/or to determine the dose of an investigational new drug in a clinical trial, allowing for the measurement or detection of a change in liver function well before any symptoms or clinically significant drug-drug interactions occur.

### SUMMARY OF THE INVENTION

The present invention is based on the observation that coproporphyrins (CPs) are early biomarkers for liver disease and damage that can be detected non-invasively in plasma or serum. Detecting CPs provides a sensitive method that can alert the patient to early-stage liver disease long before clinical symptoms present, and before routine laboratory tests alert the physician to the possibility liver disease. The present invention provides methods to assess liver function early on in disease progression and in a non-invasive manner. The methods of the invention allow a medical professional to monitor liver function during treatment, post treatment, or after withdrawal of a drug, toxin or other cause that may induce liver malfunction. The disclosed methods require only a simple blood draw to monitor the health of the liver. The disclosed methods monitor liver functions by measuring or detecting the liver's ability to remove body metabolites and other substance from the blood to bile.

The present invention describes methods of detecting and diagnosing liver function by measuring coproporphyrin I and III (CP-I and III) levels in biological samples, including but not limited to, liver tissue (e.g. a biopsy), plasma, serum, urine and feces to assess inhibition of hepatobiliary transport function, drug-induced liver injury, acute or chronic hepatocyte damage, and loss of hepatocyte total mass in the liver. The disclosed methods can also be a part of a kit used to detect liver disorders or functional changes early and in a non-invasive manner by measuring, for example, plasma levels of CP-I and CP-III.

In addition, the disclosed methods are more sensitive than currently used methods in that they detect liver malfunctions that occur in the early stages of liver disease/damage. For example, the disclosed methods can detect early stage liver inflammation or injury due to food or drug toxicity, liver damage due to disease, or liver function impairment due to drug-drug interactions. The disclosed methods allow a medical professional to monitor recovery of a person's liver function after a treatment/procedure or administration of a drug. The described methods can also be used to monitor the withdrawal of a drug that may induce liver injury or inhibit the proper functioning of hepatobiliary transport proteins in hepatocytes.

Disclosed herein are methods of monitoring CP-I and/or CP-III levels in a sample to detect a change in liver function, comprising: obtaining a sample from a subject; determining the concentration of CP-I and/or CP-III in the sample; comparing the concentration of CP-I and/or CP-III in the sample with either a normal range or a previously obtained sample from the same subject; and detecting a difference between the concentration of CP-I and/or CP-III in the sample and the normal range or previously obtained sample, wherein the difference is indicative of a change in liver function. In one embodiment, after the detecting step a diagnosis is made by a medical profession. According to various embodiments of the invention the monitoring step can be at an early stage of liver disease, at a late stage, continuously, transiently, before, during and/or after therapeutic intervention such as treatment with a drug, biologic or herbal medicine or surgical intervention.

Also disclosed herein are methods of detecting liver disease, comprising: obtaining a sample from a subject; determining the concentration of CP-I and/or CP-III in the sample; comparing the concentration of CP-I and/or CP-III in the sample with either a normal range or a previously obtained sample from the same subject; and detecting a difference, typically an increase, between the concentration of CP-I and/or CP-III in the sample and the normal range or previous sample, wherein the difference is an indication of or correlated to the presence of liver disease. In one embodiment, after the detecting step a diagnosis is made by a medical profession. In other embodiments of the invention, the liver disease is liver fibrosis, a loss of liver mass, bile duct blockage or inflammation, nonalcoholic fatty liver disease, alcoholic fatty liver disease, hepatitis, liver cancer, an infection, an immune reaction, or inflammation. In yet other embodiments of the invention, the liver disease is an acute liver disease or a chronic liver disease.

Also disclosed are methods of detecting liver injury or hepatocyte damage, comprising: obtaining a sample from a subject; determining the concentration of CP-I and/or CP-III in the sample; comparing the concentration of CP-I and/or CP-III in the sample with either a normal range or a previously obtained sample from the same subject; and detecting a difference between the concentration of CP-I and/or CP-III in the sample and the normal range or previously, wherein the change demonstrates liver injury or hepatocyte damage. In one embodiment, after the detecting step a diagnosis is made by a medical profession. In yet other embodiments of the invention, the liver disease is acute liver disease or a chronic liver disease. In other embodiments of the invention, the liver injury is drug-induced liver injury, chemical-induced liver injury, food-induced liver injury, toxin- or poison-induced liver injury, immunoreaction-induced liver injury, or alcohol-induced liver injury. In certain embodiments, the hepatocyte damage is physical hepatocyte damage or a loss of hepatocyte mass.

In addition, disclosed herein are methods of detecting hepatobiliary transport function, comprising: obtaining a sample from a subject; determining the concentration of CP-I and/or CP-III in the sample; comparing the concentration of CP-I and/or CP-III in the sample with either a normal range or a previously obtained sample from the same subject; and detecting a change in the concentration of CP-I and/or CP-III in the sample, wherein the change demonstrates a change in hepatobiliary transport function. In one embodiment, after the detecting step a diagnosis is made by a medical profession. In another embodiment of the disclosed methods, the change in hepatobiliary transport function is a change in the clearance of an endogenous compound, or a change in the clearance of an exogenous compound. In yet another embodiment, the change of hepatobiliary transport function is a result of the subject taking xenobiotics, such as a drug or a drug overdose.

Additional embodiments of any of the above-disclosed methods include wherein the detecting step is at an early stage, late stage, continually, transiently, before, during, or after therapeutic intervention as a drug treatment or surgery.

Additional embodiments of any of the above-disclosed methods include a further step comprising: determining the concentration of CP-I and CP-III in the sample, or determining the concentration of CP-I and CP-III in the sample and determining the ratio of CP-I to CP-III and/or CP-III to CP-I. Additional embodiments of any of the above-disclosed methods wherein the change in concentration of CP-I and/or CP-III is an increase, or wherein the increase is statistically significant. In some embodiments, the increase is two-fold to five-fold, five-fold to ten-fold, ten-fold to twenty-fold, twenty-fold to fifty-fold, or fifty-fold to a hundred-fold. In another embodiment of any of the disclosed methods includes wherein the change in concentration of CP-I and/or CP-III is a decrease, or wherein the decrease is statistically significant. In some embodiments, the decrease is two-fold to five-fold, five-fold to ten-fold, ten-fold to twenty-fold, twenty-fold to fifty-fold, or fifty-fold to a hundred-fold.

Additional embodiments of any of the above-disclosed methods include a treatment step. In some embodiments, the treatment comprises a change in the dose of a drug to the subject, eliminating use of a drug in the subject, administering a drug to the subject, a change in treatment of the subject, or no change in treatment of the subject.

In certain embodiments, the present invention provides methods of monitoring CP-I, CP-III or both CP-I and CP-III levels in a sample to detect a change in liver function, comprising the steps of: obtaining a sample from a subject; determining the concentration of CP-I, CP-III or both CP-I and CP-III in the sample; comparing the concentrations of CP-I, CP-III or both CP-I and CP-III in the sample with either a normal range of concentrations of CP-I and CP-III or both CP-I and CP-III in subjects with normal liver function or with CP-I and CP-III or both CP-I and CP-III concentrations determined in a previous obtained sample from the same subject; and detecting a difference between the concentration of CP-I, CP-III or both CP-I and CP-III in the sample and either the normal range or the concentrations determined in the previous sample, wherein the difference indicates a change in liver function.

The invention also provides methods of detecting a liver disease in a subject, comprising: obtaining a sample from a subject; determining the concentration of CP-I, CP-III or both CP-I and CP-III in the sample; comparing the concentration of CP-I, CP-III or both CP-I and CP-III in the sample with either a normal range of concentrations in subjects without liver disease or with a concentration determined in a previously obtained sample from the same subject; and detecting a difference between the concentration of CP-I, CP-III or both CP-I and CP-III in the sample and either the normal range or the concentration determined in the previous sample, wherein the difference indicates a liver disease.

In yet further embodiments, the invention provides methods of detecting liver injury or hepatocyte damage, comprising: obtaining a sample from a subject; determining the concentration of CP-I, CP-III or both CP-I and CP-III in the sample; comparing the concentration of CP-I, CP-III or both CP-I and CP-III in the sample with either a normal range of concentrations in subjects without liver injury or hepatocyte damage or with concentrations determined in a previously obtained sample from the same subject; and detecting a difference between the concentration of CP-I, CP-III or both CP-I and CP-III in the sample and either the normal range or the concentration detected in the previous sample, wherein the difference indicates liver injury or hepatocyte damage.

The differences determined can be an increase CP-I, CP-III or both CP-I and CP-III.

According to methods of the invention, the liver disease, change in liver function, liver injury or hepatocyte damage or diagnosis can be cirrhosis, nonalcoholic fatty liver disease, liver fibrosis, a loss of liver mass, bile duct blockage inflammation, alcoholic fatty liver disease, hepatitis, liver cancer, an infection, an immune reaction, or inflammation.

In some aspects, the liver disease is chronic; in other embodiments, the liver disease is acute.

Liver injury can for example, be drug-induced liver injury, chemically induced liver injury, food induced liver injury, poison-induced liver injury, immunoreaction-induced liver injury, or alcohol-induced liver injury.

Hepatocyte damage can, for example, be physical hepatocyte damage or a loss of hepatocyte mass.

The present invention also provides methods of assessing hepatobiliary transport function in a subject, comprising: obtaining a sample from a subject; determining the concentration CP-I, CP-III or both CP-I and CP-III in the sample; comparing the concentration of CP-I, CP-III or both CP-I and CP-III in the sample with either a normal range of concentrations determined in subjects with normal hepatobiliary transport function or with a previously obtained sample from the same subject; and detecting a change in the concentration of CP-I, CP-III or both CP-I and CP-III in the sample, wherein the change is indicative of a change in hepatobiliary transport function, thereby assessing hepatobiliary transport function in the subject.

The change in hepatobiliary transport function can be a change in the clearance of an endogenous compound or a change in the clearance of an exogenous compound.

The change in hepatobiliary transport function can be a result of exposure to a xenobiotic, such as a drug or a drug overdose.

After the detecting step in any of the methods of the invention, a diagnosis can be made by a medical professional. Thus, the methods can include the additional step of obtaining a diagnosis of a disease or condition, such as a chronic liver disease or an acute liver disease, of the subject from a medical profession. In some embodiments, the methods include the step of treating the diagnosed disease or condition.

Treating the subject for a diagnosed liver disease or liver damage can include, for example, administering an effective amount of a medication, detoxifying the liver, providing supportive care and performing a surgical procedure.

Also provided by the invention are method of treating a liver disease or liver damage in a subject, comprising the steps of obtaining a sample from a subject; determining the concentration of CP-I, CP-III or both CP-I and CP-III in the sample; comparing the concentration of CP-I, CP-III or both CP-I and CPIII in the sample with either a normal range of concentrations in subjects without liver disease or liver damage, or with a previously obtained sample from the same subject; and detecting a difference between the concentration of CP-I, CP-III or both CP-I and CP-III in the normal range and the or in the previously obtained sample, wherein a difference indicates a liver disease or liver damage; obtaining a diagnosis of liver disease or liver damage from a healthcare professional; and treating the subject for the diagnosed liver disease or liver damage.

The liver disease or liver damage according to this embodiment of the invention can be, for example, cirrhosis, nonalcoholic fatty liver disease, liver fibrosis, a loss of liver mass, bile duct blockage inflammation, alcoholic fatty liver disease, hepatitis, liver cancer, an infection, an immune reaction, or inflammation. It can be chronic or acute.

The treatment step can include, for example, administering an effective amount of a medication, detoxifying the liver, providing supportive care and performing a surgical procedure such as liver resection, ablation or liver transplant. Medications that may be used include but are not limited to: albumin, an interferon, N-acetyl cysteine, an insulin sensitizer, a statin, an antiviral, an antibiotic, a biological, an immunotherapeutic, a chemotherapeutic agent, an anti-inflammatory, a chelating agent, Silybum marianum, silymarin, silibinin, and silipide and combinations thereof.

Also provided are methods of monitoring the progression of a liver disease in a subject, comprising the steps of: obtaining a first sample from a subject with a liver disease; determining the concentration of CP-I, CP-III or both CP-I and CP-III in the sample; obtaining a second sample from the subject after a time interval; determining the concentration of CP-I, CP-III or both CP-I and CP-III in the second sample; comparing the concentration of CP-I, CP-III or both CP-I and CP-III in the second and second samples, wherein a difference in concentration of CP-I, CP-III or both CP-I and CP-III between the first and second samples is an indication of the progression of the liver disease in the subject. The time interval can be one day, one week, one month, 6 months, 12 moths, 3 years or five years. The difference in certain aspects of this embodiment is a decrease in CP-I, CP-III or both CP-I and CP-III in the second sample compared to the first sample. The process can be repeated until the liver disease resolves, with the opportunity to identify a need to change the treatment if the liver disease is not responding to treatment.

In any of the methods of the invention, the difference in concentration of CP-I, CP-III or both CPI and CP-III can be an increase. In certain aspects, the increase or other differences is a statistically significant difference. In certain aspects, the increase can be two-fold to five-fold, five-fold to ten-fold, ten-fold to twenty-fold, twenty-fold to fifty-fold, or fifty-fold to a hundred-fold.

In another embodiment, the invention provides methods of monitoring the efficacy of treating liver disease or liver damage in a subject, comprising: obtaining a first sample from a subject with liver disease or liver damage; determining the concentration of CP-I, CP-III or both CP-I and CP-III in the first sample; administering liver disease or liver damage treatment to the subject; obtaining a second sample from the subject following administering the treatment, determining the concentration of CP-I, CP-III or both CP-I and CP-III in the second sample; comparing the concentration of CP-I, CP-III or both CP-I and CP-III in the second sample with the concentration in the second sample; detecting a change in the wherein a change in concentration between the first and second samples, wherein the is indicative of the efficacy of treating the liver disease in the subject. The change can be a decrease in CP-I, CP-III or both CP-I and CP-III in the second sample compared to the first sample. The method can be repeated periodically until the liver disease or liver damage resolves, such as every day, every week, every month, every 3 months, every 6 months, or every year.

Treating the liver disease or liver damage while monitory the efficacy of treatment can be initiated and adjusted as needed. Thus, treating can include a change in the dose of a drug to the subject, eliminating use of a drug in the subject, administering a drug to the subject, a change in treatment of the subject, or no change in treatment of the subject.

Also provided are methods for screening for liver disease and/or damage, comprising: obtaining a sample from a subject during a routine medical examination; determining the concentration of CP-I, CPIII or both CP-I and CP-III in the sample; comparing the concentration of CP-I, CP-III or both CP-I and CP-III in the sample with either a normal range of concentrations in subjects without liver disease or damage or with a previously obtained sample from the same subject, wherein a difference in the concentration of CP-I, CP-III or both CP-I and CP-III in the sample and the normal range or the previous sample is an indication of liver disease or liver damage, thereby screening for liver disease or damage. In some aspects, the subject of screening can be asymptomatic. In other aspects the subject can be at risk for liver for liver disease and/or damage e.g. due to past drug or alcohol abuse, exposure to a hepatitis virus or familiar tendencies. Confirmation or differential diagnosis upon a positive test result can include additional testing or evaluation of liver disease or damage on the subject, such as blood tests, imaging tests, clinical evaluation, and biopsy. Additional follow-up blood tests include tests for ALT, AST, serum albumin, bilirubin, alkaline phosphatase, gamma glutamyl transpeptidase, 5′ nucleotidase, ceruloplasmin, alpha-fetoprotein, prothrombin time, prothrombin ratio, INR, and international normalized ratio, serum glucose, and lactate dehydrogenase. Imaging tests include ultrasound, CT or MRI scan.

In any of the methods of the invention, the sample can be selected from a plasma sample, a blood sample, a liver tissue sample, a urine sample or a fecal sample. In addition of detecting CP-I, CP-III or both CP-I and CP-III, the ratio of CP-I and CP-III can be calculated.

### BRIEF DESCRIPTION OF THE DRAWINGS

These and other features, aspects, and advantages of the present disclosure will become better understood with regard to the following description, appended claims and accompanying figures.

FIG. 1 illustrates hepatocyte organization in the liver, and excretion of coproporphyrins (CP-I and CP-III) in hepatocytes with normal liver function.

FIG. 2 illustrates the excretion of coproporphyrins in hepatocytes when hepatic transporters present on a sinusoidal or canalicular membrane are inhibited by a drug.

FIG. 3 illustrates the excretion of coproporphyrins in the liver when hepatocyte organization is disrupted or hepatocyte mass is reduced due to injury.

FIG. 4 illustrates the biosynthesis of CP-I and CP-III.

FIG. 5 is a schematic illustration of a hepatocyte comprising a sinusoidal membrane and a canalicular membrane. CP-I and CP-III are transported by liver specific membrane transporter, such as OATP and MPR2, also shown in FIG. 5. OATP and MPR2 transport endogenous molecules and drugs into the bile.

FIG. 6 is a bar graph showing CP-I levels (nM) detected in healthy subjects A and in subjects with liver disease B (Child-Pugh score 7-9).

FIG. 7 is a bar graph showing CP-III levels (nM) detected in healthy subjects A and in subjects with liver disease B (Child-Pugh score 7-9). Note that the CP-III levels in healthy subjects was below the lower limit of quantitation (LLOQ).

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this disclosure and the appended claims, the singular forms “a”, “an” and “the” include a plural reference unless the context clearly dictates otherwise. As used in this disclosure and the appended claims, the term “or” can be singular or inclusive. For example, A or B, can be A and B.

“About” as used herein means that a number referred to as “about” comprises the recited number plus or minus 1-10% of that recited number. For example, about 50 nM can mean 45-55 nM or as little as 49-51 nM depending on the situation. Whenever it appears herein, a numerical range, such as “45-55”, refers to each integer in the given range; e.g., “45-55%” means that the percentage can be 45%, 46%, etc., up to and including 55%. Where a range described herein includes decimal values, such as “1.2% to 10.5%”, the range refers to each decimal value of the smallest increment indicated in the given range; e.g. “1.2% to 10.5%” means that the percentage can be 1.2%, 1.3%, 1.4%, 1.5%, etc. up to and including 10.5%; while “1.20% to 10.50%” means that the percentage can be 1.20%, 1.21%, 1.22%, 1.23%, etc. up to and including 10.50%.

As used herein, the term “effective amount” or equivalents expressions, of e.g., a medication, therapeutic intervention or other treatment refers to that amount of the medication, therapeutic intervention or other treatment that is sufficient to effect a desired result under the conditions of use, such as the reducing, ameliorating or eliminating a disease or condition, or a symptom thereof in a subject suffering from the disease or condition. In other aspects of the invention, an “effective amount” or equivalents expressions can refer to the concentration, dosage, duration or frequency of a medication, therapeutic intervention or other treatment or a combination thereof that is sufficient to effect a desired result in reducing, ameliorating or eliminating a disease, condition, or a symptom thereof in a subject suffering from the disease or condition. An effective amount is readily determined by one of ordinary skill in the art.

An “exogenous” substance is one that does not originate from within an organism, tissue, or cell. An exogenous substance can be, for example, a pharmaceutical drug.

An “endogenous” substance is one that originates from within an organism, tissue, or cell. An endogenous substance can be, for example, a protein.

“Acute” means an abrupt onset. Acute can also mean a period of short duration, rapidly progressive, or in need of urgent care.

The term “chronic” means persisting for a period of time, typically a long time, which can be months or years, when compared to acute.

“Normal range” as used herein, refers to a range of values for a measurement such as the concentration of a biomarker, detected in healthy subjects. In one embodiment, the normal range for CP-I and/or CP-III is the range of concentrations measured in samples from two or more normal subjects who are not apparently affected by liver disease, liver damage, or liver pathology, including liver fibrosis, liver cirrhosis, hepatitis, liver failure, drug-induced liver injury, chemical-induced liver injury, food-induced liver injury, alcohol-induced liver injury, toxin- or poison-induced liver injury, immunoreaction-induced liver injury, alcohol-induced liver injury, physical hepatocyte damage, loss of hepatocyte mass, liver cancer, hepatocellular carcinoma or any other state of liver dysfunction.

Exemplary “samples” of the invention include, but are not limited to, liver tissue (for example, a biopsy, or a slice of a biopsy), plasma, serum, urine and feces.

A “subject”, as used herein, refers to a mammal, for example, a human. A subject can be a patient undergoing care of a medical professional. A subject can also be a human who is not undergoing care of a medical professional. A subject can also be a non-human mammal such as a dog, cat, or horse.

### The Liver

The liver is the second largest organ in the body and performs many essential functions. The liver mainly consists of two types of cells: Kupffer cells and hepatocytes. Kupffer cells are phagocytic cells that form the lining of the sinusoids of the liver and are involved in the breakdown of red blood cells. The function of Kupffer's cells is to filter bacteria and other small foreign proteins out of the blood. Hepatocytes are epithelial parenchymatous cells of the liver. Hepatocytes make up 70-85% of the mass of the liver. Hepatocytes are involved in: protein synthesis; transformation of carbohydrates; syntheses of cholesterol, bile salts and phospholipids; detoxification, modification, and excretion of exogenous and endogenous substances; and the initiation of formation and secretion of bile.

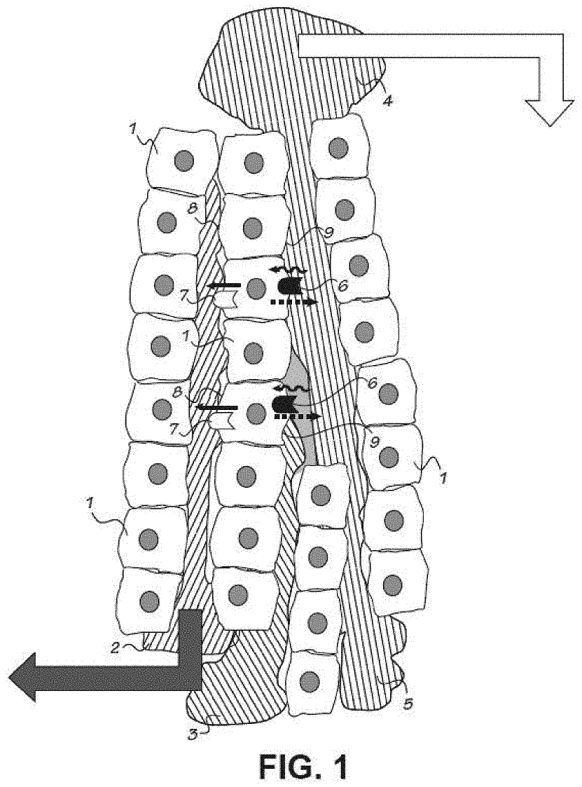

FIG. 1, illustrates transport in a normally functioning liver. For reference, this figure illustrates the relative positions of hepatocytes 1, a bile duct 2, the hepatic portal vein 3, central vein 4, arteriole 5, sinusoidal uptake transporter 6, canalicular efflux transporters 7, canalicular membrane 8, sinusoidal membrane 9. Referring to FIG. 1, hepatocytes 1 are polarized with distinct sinusoidal domains and apical domains that are separated by tight junctions, which sustain two countercurrent flow systems-bile secretion (indicated by large filled arrow) and blood circulation (indicated by large unfilled arrow). The hepatobiliary transporters rely on the unique tissue architecture of the liver to remove many endogenous and exogenous molecules from systemic circulation, for example, from plasma to bile. Endogenous and exogenous molecules are taken up by specific sinusoidal membrane transport proteins 6. Following their uptake, these molecules can be further metabolized within the hepatocytes. These molecules or their metabolites can also be released back into the systemic circulation across the sinusoidal membrane 9 (broken arrows), or transported across the bile canalicular membrane 8 of a hepatocyte into the bile via apical membrane transport proteins 7.

In a normally functioning liver, about 70% of CP-I and about 30% of CP-III are eliminated in the bile. The remaining CP-I and CP-III are excreted in the urine.

To remove CP-I and CP-III from plasma, membrane transporter proteins (as shown in FIG. 1 and FIG. 5) must function properly. The movement or liver clearance of CP-I and CP-III across the sinusoidal 9 or canalicular 8 membranes of hepatocytes is shown in FIG. 1 follows the directions of broken and solid arrows.

Hepatobiliary coproporphyrins clearance relies on: 1) proper functioning of transporter proteins in the hepatocytes; 2) healthy liver structure and overall organization of the hepatocytes; and 3) total hepatocyte number in the liver. Reduction of hepatobiliary elimination causes an increase of coproporphyrins levels in the systemic circulation, thus, the levels of coproporphyrins in plasma reflects liver function, for example, either liver injury or functional impairment of transport proteins (as can be present in acute or chronic liver infection or inflammation).

Regarding “total hepatocyte number”, usually normal liver contains a certain number of hepatocytes per gram tissue, for example 1012 cells per gram liver. Some diseases, for example, liver fibrosis, can have the same mass of the liver by weight, but the functional hepatocyte number is lost, for example, only 108 cells per gram liver.

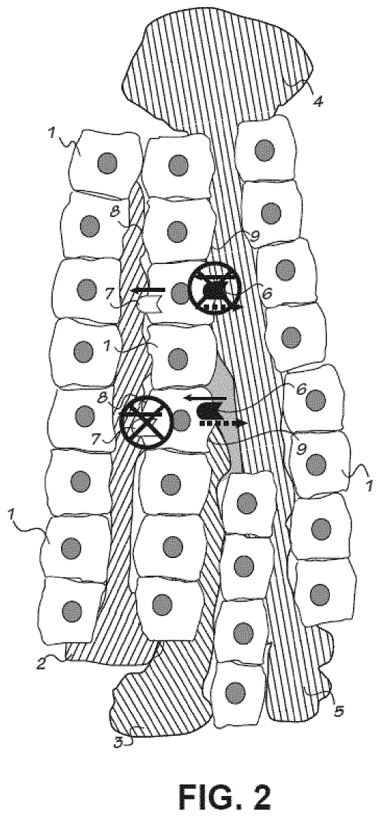

FIG. 2 shows that impairment of membrane transport proteins by drug or liver damage (as shown by two circles each with an “X”) on either the sinusoidal membrane, for coproporphyrin uptake, or on the canalicular membrane, for coproporphyrin efflux, or both, can result in reduced coproporphyrin clearance from systemic circulation, and elevated coproporphyrin levels in plasma.

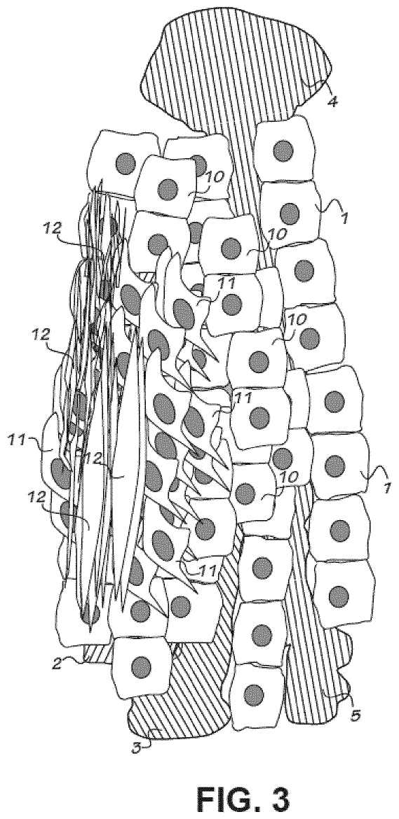

FIG. 3 shows that plasma clearance of coproporphyrins can be disrupted by abnormal organization of hepatocytes 10 in the liver (for example, the lining of the cells), growth of fibroblasts 11 and fibrosis 12 (excess fibrous connective tissue or scarring) due to inflammation, or other liver diseases.

The rate of coproporphyrin removal from systemic circulation is decreased by inhibition of hepatobiliary transport proteins (FIG. 2), or by liver damage, injury, or loss of functional hepatocyte per unit liver mass (FIG. 3). The decreased rate of coproporphyrin removal results in an increase of coproporphyrin levels in plasma. As a result, coproporphyrin levels present in plasma are biomarkers that reflect liver function.

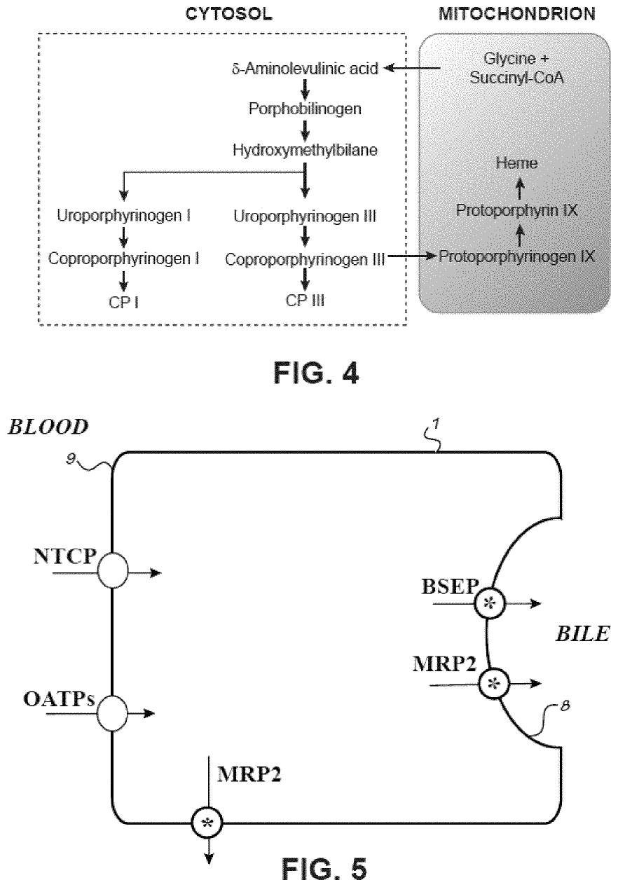

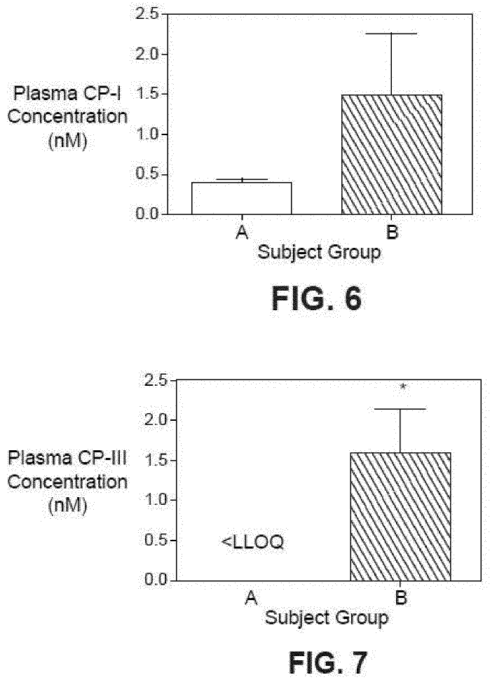

CP-I and CP-III are formed as byproducts of heme biosynthesis. As shown in FIG. 4, heme biosynthesis is initiated by the formation of 5-aminolevulinic acid (ALA) from glycine (Gly) and succinyl-CoA, which takes place mainly in the matrix of mitochondria of human hepatocytes. ALA enters the cytosol, where it forms porphobilinogen (PBG), catalyzed by ALA dehydratase. PBG is then polymerized to generate a linear tetrapyrrole hydroxymethylbilane (HMB). HMB readily cyclizes to form uroporphyrinogen I and III. The uroporphyrinogens undergo decarboxylation to form coproporphyrinogen I and III. Coproporphyrinogen III is transported into the mitochondria and converted into protoporphyringen IX for heme synthesis. Both uroporphyrinogen I and III also are also converted into CP-I and CP-III, respectively, under chemical reactions, for example, exposure to light. CP-I and CP-III do not undergo further metabolism and are stable in the plasma.

In systemic circulation, CP-I and CP-III are not enzymatically digested, and are removed from the body via the bile and urine as intact molecules. In the liver, removing CP-I and CP-III require the presence of specific hepatic membrane transport proteins. The transport proteins present in the sinusoidal membrane, take up CP-I and CP-III into the hepatocyte, while other transport proteins extrude CP-I and CP-III into the bile, across the canalicular membrane.

### Hepatocytes

A hepatocyte is a cell of the main parenchymal tissue of the liver. Hepatocytes make up approximately 70-85% of the liver's mass.

### Hepatobiliary Transporters

Sinusoidal membrane transporters and canalicular membrane transporters, include, for example, NTCP, OATPs, MRP3, BSEP, and MRP2 as shown in FIG. 5.

### Hepatic Transport Proteins

The liver is a major contributor to first-pass elimination of orally administered drugs as well as for plasma clearance of systemically distributed drugs. The amount of blood filtered by the liver per time unit is considerable: it accounts for approximately 1500 ml/min. The major part, approximately 70-80%, originates from the portal vein, whereas the remaining percentage originates from the hepatic artery. Both influxes lead to the hepatic sinuses, where drugs may be extracted by hepatocytes, metabolized and subsequently exported from the cell. Uptake transporters of the solute carrier family facilitate the hepatocellular uptake of their substrates. Export transporters at the canalicular or sinusoidal membrane, mostly energy-dependent transporters of the ATP-binding cassette transporters, contribute to the export of drugs and their metabolites into the bile or back into the blood.

Exemplary hepatic drug transporters can be solute carriers (OATPs, OAT2, OCT1, NTCP) or ABC transporters (MDR1, MRPs, BCRP, BSEP). Many of these transporters lack selective substrates or inhibitors, have multiple binding sites, making it hard to understand their physiological role (Funk, “The role of hepatic transporters in drug elimination,” Expert Opin Drug Metab Toxicol. 2008 April; 4(4):363-79. doi: 10.1517/17425255.4.4.363).

### Advantages of the Disclosed Methods

The disclosed methods use CP-I and CP-III as highly sensitive markers to detect changes in liver function. Both CP-I and CP-III can detect a change in liver function, even if no physical change or damage can be seen in the liver tissue itself and no clinical symptoms are reported by a patient. Existing enzymatic assays, such as ALT or AST described above, require the liver tissue to be damaged to be able to measure the concentrations of the two enzymes. In other words, the disclosed methods are more sensitive because they can detect a change in liver function prior to the appearance of damaged liver tissue.

The disclosed invention is a method of detecting asymptomatic chronic liver diseases, such as alcoholic or nonalcoholic fatty liver disease, primary and secondary liver cancer, hemochromatosis, Gilbert's syndrome, portal hypertension, viral infections in the liver (hepatitis A, B, C), liver cirrhosis, hepatic necroinflammation and fibrosis.

### Early Stage Detection or Monitoring of CP-I and/or CP-III

The disclosed methods of detecting or monitoring CP-I and/or CP-III can be used for early-stage diagnosis or detection of changes in liver function.

Early stage can be, for example, one week to 120 months prior to presenting symptoms of loss of liver function or change in liver function or detection by routine methods.

Early stage can be any time from the period of initiation of a change in liver function to the presentation of symptoms or detection by routine biochemistry methods.

Early stage can be any time after administration of a drug to a patient, for example, greater than 30 minutes post-administration of the drug, greater than one hour post-administration of a drug, or to up to 2 years post-administration of a drug.

Early stage can be prior to detection in a change (for example, a decrease) in bilirubin concentration in the blood.

Early stage can be immediately after a drug is administered to a patient and the drug's effects on modulating liver function are detected.

### Constant or Transient Detecting or Monitoring of CP-I and/or CP-III

The disclosed methods of monitoring or detecting CP-I and/or CP-III can be used, for example, to monitor the progression of a drug regimen, monitor the effectiveness of a treatment plan or drug, monitor the inhibition of hepatic transport function by a drug, monitor the progress of liver disease/injury, or monitor the recovery of liver function. The monitoring can be constant, or transient. For example, the monitoring can be every three hours for one week or one time.

### Later Stage Detection or Monitoring of CP-I and/or CP-III

The disclosed methods of detecting or monitoring CP-I and/or CP-III can be used for late stage diagnosis in which although liver function may be viewed or detected by known methods, the disclosed methods are more sensitive in monitoring liver function and are therefore more effective in treating the patient.

### Coproporphyrins CP-I and CP-III

The liver is a key organ in porphyrin metabolism and excretion. CP-I and III are porphyrin metabolites arising from heme synthesis. Porphyrins are pigments found in both animal and plant life. CPI and III are tetrapyrrole dead-end products from the spontaneous oxidation of the methylene bridges of coproporphynogen, arising from heme synthesis and are secreted in feces and urine. The chemical structures of CP-I AND CP-III (obtained from PubChem) are shown below.

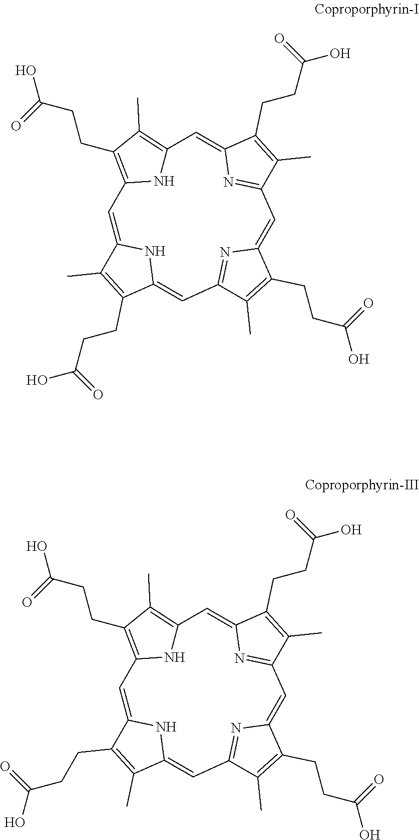 [SMILES: CC1=C(CCC(=O)O)c2cc3nc(cc4[nH]c(cc5[nH]c(cc1n2)c(CCC(=O)O)c5C)c(CCC(=O)O)c4C)C(CCC(=O)O)=C3C ; CC1=C(CCC(=O)O)c2cc3nc(cc4[nH]c(cc5[nH]c(cc1n2)c(C)c5CCC(=O)O)c(CCC(=O)O)c4C)C(CCC(=O)O)=C3C]

### Coproporphyrin-III

The physiological functions of CP-I and CP-III are not fully understood. CP-I and CP-III are both cleared from the liver through hepatobiliary transporters present in hepatocytes, such as OATPs and multidrug resistance protein2 (MRP2). These two classes of membrane spanning proteins, transport endogenous molecules and drugs into the bile.

Impairment of hepatobiliary transporters, or hepatocyte damage or loss of hepatocyte numbers in the liver, due to, for example, liver fibrosis or non-alcoholic fatty liver disease (NASH) increases the levels of CP-I and CP-III in plasma or serum. In other words, disruption or loss of the normal function of hepatobiliary transport proteins affects the clearance of CP-I and CP-III from the plasma (blood) to the bile. If hepatocyte injury occurs, both sinusoidal and canalicular membrane transport proteins can be damaged/affected. Drugs can also inhibit the proper transport of CP-I and CP-III across a transporter protein, such as OATPs.

Any damage or injury to liver cells, or loss of liver mass or loss of hepatocyte numbers in the liver will change (increase) the concentrations of CP-I and CP-III in blood (plasma and serum). Therefore, CP-I and CP-III levels in blood can be used as biomarkers for liver function. CP-I and CP-III levels in blood, can be used as biomarkers to monitor liver function in a diseased liver, or to measure the pathological effects of a drug or toxin (acute, chronic, or spontaneous) even without the presence of active cell damage, which is required for many current tests of liver function such as ALT or AST.

CP-I and CP-III have not been used to measure liver function. However, measurement of urinary PBG or total porphyrin levels in urine or plasma is used in the clinical diagnosis of porphyria, to diagnose several genetic human diseases, and to determine the level of damage to red blood cells due to, for example, lead or arsenic toxicity.

### Clearance Rate of CP-I and/or CP-III

There can be, for example, equal or greater than a two-fold increase in the plasma level of CP-I and/or CP-III between a healthy subject and an unhealthy patient with liver problems (for example, damaged or diseased liver). In this example, the plasma level of a patient is compared to that of a healthy person, or range of healthy persons.

There can be, for example, equal or greater than a two-fold increase in the plasma level of CP-I and/or CP-III in a patient as compared to that of the same patient in a different time. In this example, the plasma level of a patient is compared to that of the same patient at a different time.

Greater than about a two-fold increase of plasma CP-I and/or CP-III suggests a loss of hepatocyte function.

In the disclosed methods, greater than about a two-fold increase of plasma CP-I and/or CP-III can be measured, greater than about a three-fold increase of plasma CP-I and/or CP-III can be measured, greater than about a four-fold increase of plasma CP-I and/or CP-III can be measured, or greater than about a five-fold increase of plasma CP-I and/or CP-III can be measured.

### CP-I/CP-III Ratio

The body primarily makes the isomer III type of porphyrinogens, because only these are precursors of heme. But some isomer I coproporphyrinogens are made in small amounts and are excreted. High-performance liquid chromatography (HPLC) or Ultra Performance Liquid Chromatography (UPLC) can be used to detect the different isomers. The isomer I coproporphyrinogens predominate in urine (as well as erythrocytes, plasma and feces) in congenital erythropoietic porphyria (CEP). In addition to the sample level of each individual CP-I and CP-III, the ratio of CP-III over CP-I, or vice versa, can also be measured by the disclosed methods to monitor liver function.

### Porphyria

Porphyrias are metabolic disorders in which the clinical manifestations are attributable to decreased activity of a specific enzyme(s) in the heme synthesis pathway (FIG. 4), associated with characteristic patterns of overproduction of specific heme precursors and resultant accumulation in certain tissues. Each enzyme deficiency results in a predictable accumulation of the preceding heme precursor(s), and overall production of heme is generally preserved. Porphyrias, when clinically active, and in some cases even when latent or in clinical remission, are characterized by high levels of heme precursors in blood, urine, and/or stool. Most types of porphyria are inherited conditions; however, porphyria cutanea tarda, is known to occur in acquired or inherited manner. See Medical Treatment Guidelines posted online October 1995 by Washington State Department of Labor and Industries.

Measurement of urine porphobilinogen (PBG), urine total coproporphyrin, fecal coproporphyrin, blood total porphyrins or urine uroporphyrin has been reported in Porphyria patients. However, the measurement can be compromised by various factors including limitation of analytical methods, laboratory-specific reference ranges, blood lead intoxication, specimen integrity and the individual laboratory performing the test. With the wide availability of genomic sequencing, such genetic testing has become the conventional and preferred method for diagnosing Porphyrias.

### Hepatitis

Hepatitis is a disease characterized by inflammation of the liver. Hepatitis is an inflammation or infection of the liver. It may be caused by drugs, alcohol use, or certain medical conditions. But in most cases, it's caused by a virus. This is known as viral hepatitis, and the most common forms are hepatitis A, B, and C. Sometimes there are no symptoms of hepatitis in the first weeks after infection—the acute phase. But when they happen, the symptoms of types A, B, and C may include fatigue, nausea, poor appetite, belly pain, a mild fever, or yellow skin or eyes (jaundice). When hepatitis B and C become chronic, they may cause no symptoms for years. By the time there are any warning signs, the liver may already be damaged. Chronic hepatitis can quietly attack the liver for years without causing any symptoms. Unless the infection is diagnosed, monitored, and treated, many of these people will eventually have serious liver damage.

### Hepatocellular Carcinoma

Hepatocellular carcinoma (HCC) is the fifth leading cause of death from cancer in men, the seventh leading cause of death from cancer in women, and the fastest rising cause of cancer mortality worldwide. The majority of patients present at an advanced stage when treatment options are very limited and, consequently, HCC carries a dismal prognosis (overall median survival of 14 weeks, 1-year survival of 13%). Current screening strategies that rely on AFP and ultrasound are widely accepted but have only modest diagnostic accuracy with sensitivity rates between 25% and 65%. There is an urgent need to discover and implement better diagnostic tools for this malignancy that may permit earlier and more accurate detection. (See G. P., et al., International Journal of Hepatology, Volume 2012, Article ID 437508, 4 pages, doi:10.1155/2012/437508).

### Plasma

Plasma is the colorless fluid part of blood, lymph, or milk, in which corpuscles or fat globules are suspended.

### Serum

The blood serum of an animal can be used as a diagnostic agent. Serum is the liquid portion of the blood after it has been allowed to clot. It is free of clotting proteins but contains the clotting metabolites that result from the clotting process. It is a cleaner sample typically free of cells and platelets because they are trapped in the fibrin meshwork of the clot. Plasma, on the other hand, is the liquid portion of blood that has been prevented from clotting and is more reflective of the blood as it circulates in the body. Though it has an advantage over serum in that testing is not delayed by waiting 30 minutes for a clot to form, it is typically contaminated by platelets and cellular elements that have the potential to alter analytical results.

### Collection of Serum and Plasma

Serum and plasma samples can be collected in evacuated tubes with a gel barrier, which separates the sample from the cellular elements that could impact analytical values. Centrifugation should occur within two hours of collection with light protection. Gels are thixotropic substances that liquefy upon centrifugation to move rapidly into place based on specific gravity. These inert gel polymers have a specific gravity that is between that of serum/plasma and the cellular portion of blood, typically around 1.04 g/cm3. With a serum sample, the gel moves up the side wall of the tube around the clot to settle into position. With plasma, this process involves free cellular elements moving down through the gel as it migrates up making it more challenging to achieve clean separation.

It is important that sample centrifugation is sufficient to achieve platelet-poor plasma as required for most routine assays. This is typically defined as a platelet count of less than 10,000. Other settings, i.e. higher g-force or less time, may also result in platelet-poor plasma but these setting should be validated to ensure that these changes do not result in increased platelets and cellular elements being trapped above the gel or have any impact on analysis.

### Routine Biochemistry

Patients can have a liver function test profile performed that includes measuring the levels of one or more of the following: plasma albumin, bilirubin, alkaline phosphatase (ALP), gamma-glutamyl transpeptidase (GGT), alanine aminotransferase (ALT) and aspartate aminotransferase (AST). In addition, iron study profiles can be performed consisting of serum ferritin, iron and transferrin, and transferrin saturation can be calculated.

### Measurement of Coproporphyrins

The disclosed methods can be used as a method of extracting CP-I and/or CP-III from plasma, urine or feces, by concentrated hydrochloric acid or formic acid prior to the CP-I or CP-III detection.

CP-I and/or CP-III in samples can be measured or detected spectrofluorometrically and expressed as nmol/L.

CP-I and/or CP-III can be measured or detected in samples using reverse phase, high-performance liquid chromatography (HPLC) or UPLC with fluorescence detection or mass spectrometry.

Other methods known to one of skill in the art to measure or detect CP-I and/or CP-III can be used in the disclosed methods. In addition, any future methods discovered to measure or detect CP-I and/or CP-III can also be used in the disclosed methods.

### Alternative Disclosed Methods

The disclosed methods can be used to measure CP-I and/or CP-III levels in plasma or serum, using a simple blood draw, or a urine or fecal sample, to detect early changes in liver function and malfunction that cannot be detected by routine screening, for example, enzyme levels.

The disclosed methods can be used to measure the inhibition or alternation of hepatobiliary transport of CP-I and/or CP-III levels in plasma, serum, urine and feces.

The disclosed methods can be used to measure drug-induced liver injury.

The disclosed methods can be used to measure the clearance changes of a drug from the liver.

The disclosed methods can be used to detect bile duct hyperplasia.

The disclosed methods can be used to measure acute or chronic hepatocyte damage (for example, inflammation, toxin, abnormal organization, disruption, or immunoreaction).

The disclosed methods can be used to measure the loss of hepatocyte total mass in a liver.

The disclosed methods can be used as a method of extracting CP-I and/or CP-III from plasma, urine or feces, by concentrated hydrochloric acid or formic acid prior to the CP-I or CP-III detection.

The disclosed methods can be used as a non-invasive and sensitive method of measuring CP-I and CP-III levels in plasma, urine, or feces.

The disclosed methods can be used as non-invasive and sensitive diagnostic method for the detection of changes in CP-I and CP-III that are present in early stage liver disease prior to the appearance of symptoms.

The disclosed methods can be used as a method of monitoring liver transport clearance posttreatment of a drug (both short term and/or long term; continuous or discontinuous).

The disclosed methods can be used to determine whether to withdraw the use or limit the use of a drug.

The disclosed methods can be to diagnose liver function or dysfunction, or to detect functional liver changes early on during disease progression.

The disclosed methods can be used as a method of early diagnosis of liver function impairment.

The disclosed methods can be used to monitor liver detoxification function.

The disclosed methods can be used to determining hepatic transporter inhibition or alternation by a drug.

The disclosed methods can be used to monitor inhibition of hepatobiliary transport on a sinusoidal or canalicular membrane or both sinusoidal and canalicular membrane of a hepatocyte by a drug.

The disclosed methods can be used to detect (diagnose) a change in hepatocyte organization (lining) or loss of hepatocyte number in a liver, for example, due to a liver tumor.

The disclosed methods can be used to prevent or reduce drug-induced liver injury or loss of function.

The disclosed methods can be used to continuously monitor or transiently monitor liver function.

The disclosed methods can be used to determine the clearance rate of CP-I and/or CP-III from the liver.

The disclosed methods can be used to monitor a patient's liver function after a liver transplantation.

The disclosed methods can be used monitor the health of a liver of a patient diagnosed with Hepatitis, for example, Hepatitis C or Hepatitis B.

The disclosed methods can be used to monitor liver function after liver injury.

### EXAMPLES

The following examples are intended to provide illustrations of the application of the present disclosure. The following examples are not intended to completely define or otherwise limit the scope of the disclosure.

One of skill in the art will appreciate that many other methods known in the art may be substituted in lieu of the ones specifically described or referenced herein.

### Example 1: Detection of CP-I and CP-III in Normal Subjects and Patient Selection

The Child-Pugh Score was introduced in 1973 for estimation the risk of operative mortality in patients with bleeding esophageal varices. Since then, it has been modified as a non-invasive indicator to assess prognosis in patients with chronic liver disease and cirrhosis. The score is constructed by five factors; they are total bilirubin level, serum albumin, prothrombin time or international normalized ratio (INR), degree of ascites and degree of hepatic encephalopathy.

It has been noted that Critics of the Child-Pugh score relied on clinical assessment, which may result in inconsistency in scoring. In addition, its broad classifications of disease are impractical when determining priority for liver transplantation.

Subjects who were ≥18 years old and have provided prior written informed consent to participated in this study. Group A: six healthy volunteers. Group B: six liver disease subjects who are diagnosed by a licensed physician with Child-Pugh Score (CPT) of 7-9.

Subjects were excluded in the studies: subjects who were younger than 18 years of age or older than 65 years of age; and those who had a history of Blood Borne Diseases including HIV, HCV, HBV, and syphilis; patients who were pregnant, lactating, or generally not healthy enough for blood donation; subjects who have not provided prior written informed consent to participate. Patient demographics are listed in Table 1.

TABLE 1
Patient demographics

| | | | Date of | CPT Score | | | | | Smoking |
|---|---|---|---|---|---|---|---|---|---|
| Sample ID | Date Collected | Diagnosis | Diagnosis* | & Date* | Age | Gender | Race | Ethnicity | Status |
| LIV1024004 | Aug. 22, 2018 | Nonalcoholic | Jan. 7, 2016 | CPT of 7 on | 53 | F | African | Non Hispanic | Non |
| | | steatohepatitis | | Jul. 2, 2018 | | | American | | Smoker |
| LIV1024005 | Aug. 22, 2018 | Cirrhosis | Oct. 6, 2015 | CPT of 9 on | 46 | F | Caucasian | Non-Hispanic | Non- |
| | | | | Jul. 2, 2018 | | | | | Smoker |
| LIV1024008 | Aug. 27, 2018 | Cirrhosis | May 7, 2018 | CPT of 7 on | 54 | M | Caucasian | Non-Hispanic | Non- |
| | | | | Jul. 2, 2018 | | | | | Smoker |
| LIV1024010 | Aug. 28, 2018 | Nonalcoholic | Jun. 25, 2018 | CPT of 9 on | 58 | F | African | Non-Hispanic | Non- |
| | | steatohepatitis | | Jul. 2, 2018 | | | American | | Smoker |
| LIV1024011 | Aug. 29, 2018 | Cirrhosis | Sep. 11, 2018 | CPT of 9 on | 38 | M | Caucasian | Non-Hispanic | Current |
| | | | | Sep. 11, 2018 | | | | | Smoker |
| LIV1024012 | Sep. 4, 2018 | Nonalcoholic | Feb. 8, 2017 | CPT of 7 on | 44 | F | Caucasian | Non-Hispanic | Current |
| | | steatohepatitis | | Jul. 13, 2018 | | | | | Smoker |
| NOR1024001 | Sep. 5, 2018 | Normal | NA | NA | 41 | M | Caucasian | Non-Hispanic | Non- |
| | | | | | | | | | Smoking |
| NOR1024002 | Sep. 5, 2018 | Normal | NA | NA | 51 | F | Caucasian | Non-Hispanic | Non- |
| | | | | | | | | | Smoking |
| NOR1024003 | Sep. 5, 2018 | Normal | NA | NA | 52 | F | Caucasian | Non-Hispanic | Non- |
| | | | | | | | | | Smoking |
| NOR1024004 | Sep. 5, 2018 | Normal | NA | NA | 35 | F | Caucasian | Non-Hispanic | Non- |
| | | | | | | | | | Smoking |
| NOR1024005 | Sep. 5, 2018 | Normal | NA | NA | 48 | F | Caucasian | Non-Hispanic | Non- |
| | | | | | | | | | Smoking |
| NOR1024006 | Sep. 5, 2018 | Normal | NA | NA | 45 | F | Caucasian | Non-Hispanic | Non- |
| | | | | | | | | | Smoking |

*NA, Not applicable

TABLE 2
CPT Score and Patient Prognosis

| Factor | 1 point | 2 points | 3 points |
|---|---|---|---|
| Total bilirubin | <34 | 34-50 | >50 |
| (μmol/L) | | | |
| Serum albumin | >35 | 28-35 | <28 |
| (g/L) | | | |
| PT INR | <1.7 | 1.71-2.30 | >2.30 |
| Ascites | None | Mild | Moderate to Severe |
| Hepatic | None | Grade I-II | Grade III-IV |
| encephalopathy | | (or suppressed | (or refractory) |
| | | with medication) | |
| | Class A | Class B | Class C |
| Total CPT points | 5~6 | 7~9 | 10~15 |
| 1-year survival | 100% | 80% | 45% |

### Plasma Sample Collection

Blood was collected in 2×10 mL labeled K2 EDTA Vacutainer tube and then immediately inverted 10 times gently. The tubes were then centrifuged for 15 minutes at 1500 rcf at 4° C. with light protection. The plasma was transferred to cryovials and stored in a dark −70° C. freezer until shipment for analysis.

### Sample Preparation for Coproporphyrin-I (CP-I) and III (CP-III) Analysis

An aliquot of 50 μL of plasma sample was treated with 0.1% perchloric acid (10:1 v:v) and mixed with 100 μL of acetonitrile containing internal standard (Terfenadine). The mixture was vortexed on a shaker for 15 minutes and subsequently centrifuged at 4000 rpm for 15 minutes. An aliquot of 70 μL of the supernatant was mixed with 70 μL of water for the injection to the LC/MS. Calibration standards and quality control samples were prepared by spiking the CP-I and CP-III into 2.5% BSA in water (treated with 0.1% perchloric acid) and then processed with the unknown samples. CP-I and CP-III calibration standards were prepared at molar concentrations of 0, 0.05, 1, 10, 20, 100 nM each. Separation of CP-I and CP-III was achieved using Shimadzu LC30AD system consisting of a Phenomenex Luna Phenyl-Hexyl column (100×2 mm, 5μ). The system was held at 40° C. and with a constant flow of 0.5 mL/min using a gradient comprising of Mobile phase A (10 mM ammonium formate in water with 0.1% formic acid) and mobile phase B (acetonitrile). The Liquid Chromatography (LC) program starting conditions are 25% B. At 1 minutes % B is increased to 50% over 10 min, and then further increased to 95% in 1 min, maintaining 95% B for 0.8 min and then decreasing to starting conditions (25% B) in 0.2 min and maintaining this until 13.5 min. A Triple Quad™ 5000 mass spectrometer was operated in positive electrospray ionization in MRM mode with the transitions of m/z 655.26>596.3 for CP-I and CP-III, m/z 472.183>436.126 for terfenadine (IS). The chromatographic peaks were processed using Analyst® software (version 1.6.2, AB SCIEX). The lower limit of quantitation (LLOQ) of 0.05 nM was achieved for both analytes.

### Results

Mean plasma concentration of CP-I and CP-III are shown in FIG. 1 and FIG. 2. The mean concentration of CP-I in plasma was increased about 3.8-fold in subjects with liver diseases with Child-Pugh Score of 7-9, compared to the healthy subjects. The maximum fold change of CP-I in subjects with liver disease was 10.6-fold over the mean concentration of CP-I in healthy subjects (FIG. 1). The mean plasma concentration of CP-III was 0.16±0.05 nM in subjects with liver disease, while the CP-III concentration in plasma was below the Lower Limit of Quantification (<LLOQ) in the group of healthy subjects.

1-47. (canceled)

48. A method of detecting a change in liver function in a subject comprising:
a) obtaining a biological sample from the subject;
b) measuring the concentration of coproporphyrin I (CP-I) and/or coproporphyrin III (CP-III) in the biological sample;
c) comparing the concentration of CP-I and/or CP-III in the biological sample from the subject with either the concentration CP-I and/or CP-III measured in subjects with normal liver function or the concentration of CP-I and/or CP-III measured previously in the same subject;
d) detecting a change in liver function by comparing the concentration of CP-I and/or CP-III in the biological sample from the subject with either the concentration CP-I and/or CP-III in subjects with normal liver function or the concentration of CP-I and/or CP-III measured previously in the same subject; and
e) wherein steps a) to d) are repeated for continuously monitoring

49. The method of claim 48 wherein the change in liver function in the subject is used to detect liver disease, liver injury and/or to monitor the disruption of hepatobiliary transport.

50. The method of claim 49 wherein liver disease is selected from cirrhosis, nonalcoholic fatty liver disease, liver fibrosis, a loss of liver mass, bile duct blockage, alcoholic fatty liver disease, hepatitis, liver cancer, an infection, an immune reaction, and/or inflammation.

51. The method of claim 49 wherein liver injury is selected from drug-induced liver injury, the disruption of hepatobiliary transport, chemically induced liver injury, food-induced liver injury, poison-induced liver injury, immunoreaction-induced liver injury, and/or alcohol-induced liver injury.

52. The method of claim 48 wherein the change in liver function in the subject is an increase or a decrease in CP-I and/or CP-III concentrations and/or their concentration ratios in the biological sample when compared to either the concentration CP-I and/or CP-III and/or their concentration ratios measured in subjects with normal liver function or the concentration of CP-I and/or CP-III and/or their concentration ratios measured previously in the same subject.

53. The method of claim 52 wherein the change in CP-I and/or CP-III concentrations in the biological sample is greater than a two-fold or a statistically significant.

54. The method of claim 53 wherein the biological samples are, but not limited to, a plasma, blood, serum, urine, organ tissue, or fecal sample.

55. The method of claim 48 wherein the biological samples are, but not limited to, a plasma, blood, serum, urine, organ tissue, or fecal sample.

56. The method of claim 48 further comprising measuring in the subject the levels of one or more of the following: plasma albumin, direct or indirect bilirubin, alkaline phosphatase (ALP), gamma-glutamyl transpeptidase (GGT), alanine aminotransferase (ALT) and aspartate aminotransferase (AST).

57. A method of monitoring the progression of a liver disease in a subject comprising:
a) obtaining a first biological sample from the subject;
b) measuring the concentration of coproporphyrin I (CP-I) and/or coproporphyrin III (CP-III) in the first biological sample;
c) obtaining a second biological sample from the subject after a time interval;
d) measuring the concentration of CP-I and/or CP-III in the second biological sample;
e) comparing the concentration of CP-I and/or CP-III and/or their concentration ratios in the first and second biological samples wherein a difference in concentrations of CP-I and/or CP-III and/or their concentration ratios is used to monitor the progression of the liver disease in the subject; and
f) wherein steps a) through e) are repeated for continuously monitoring the progression of the liver disease.

58. The method of claim 57, wherein the time interval is selected from the group consisting of one day, one week, one month, six months, twelve months, three years or five years.

59. The method of claim 57, wherein the first and second biological samples are, but not limited to, plasma, blood, serum, urine, organ tissue, or fecal samples.

60. The method of claim 57, wherein the steps of:
a) obtaining a first biological sample from the subject;
b) measuring the concentration of coproporphyrin I (CP-I) and/or coproporphyrin III (CP-III) in the first biological sample;
c) obtaining a second biological sample from the subject after a time interval;
d) measuring the concentration of CP-I and/or CP-III in the second biological sample;
e) comparing the concentration of CP-I and/or CP-III and/or their concentration ratios in the first and second biological samples wherein a difference in concentrations of CP-I and/or CP-III and/or their concentration ratios is used to monitor the progression of the liver disease in the subject; and
f) wherein steps a) through e) are repeated to continuously monitor the progression of the liver disease.

61. A method of monitoring the efficacy of treating liver disease, drug-induced liver injury and/or the disruption of hepatobiliary transport in a subject in need of comprising:
a) obtaining a first biological sample from the subject;
b) measuring the concentration of coproporphyrin I (CP-I) and/or coproporphyrin III (CP-III) in the first biological sample;
c) administering a liver disease and/or liver injury treatment to the subject or administering a drug or drugs that potentially disrupt hepatobiliary transport to the subject;
d) obtaining a second biological sample from the subject after a time interval following administering the liver disease and/or liver injury;
e) measuring the concentration of CP-I and/or CP-III in the second biological sample;
f) comparing the concentration of CP-I and/or CP-III and/or their concentration ratios in the first and second biological samples wherein a difference in concentrations of CP-I and/or CP-III and/or their concentration ratios is used to monitor the efficacy of treating of the liver disease and/or liver injury, or the changes of hepatobiliary transport in the subject; and
g) wherein steps a) through f) are repeated for continuously monitoring

62. The method of claim 61, wherein the time interval is selected from the group consisting of one day, one week, one month, six months, twelve months, three years or five years after administering a drug or multiple drugs to treat liver disease and/or liver injury treatment.

63. The method of claim 61, wherein the first and second biological samples are, but not limited to plasma, blood, serum urine, organ tissue, or fecal samples.

64. The method of claim 61, wherein the efficacy of treating the liver disease and/or liver injury in the subject is determined by a decrease in concentrations of CP-I and/or CP-III and/or change of their concentration ratios in the second biological sample compared to the first biological sample.

65. The method of claim 61, wherein the steps of:
a) obtaining a first biological sample from the subject;
b) measuring the concentration of coproporphyrin I (CP-I) and/or coproporphyrin III (CP-III) in the first biological sample;
c) administering a liver disease and/or liver injury treatment and/or a drug or multiple drugs that potentially disrupt hepatobiliary transport to the subject;
d) obtaining a second biological sample from the subject after a time interval following administering the liver disease and/or liver injury;
e) measuring the concentration of CP-I and/or CP-III in the second biological sample; and
f) comparing the concentration of CP-I and/or CP-III and/or their concentration ratios in the first and second biological samples wherein a difference in concentrations of CP-I and/or CP-III and/or their concentration ratios is used to monitor the efficacy of treating of the liver disease and/or liver injury and/or the disruption of hepatobiliary transport in the subject.
g) wherein steps a) through f) are repeated until the liver disease, the liver injury and/or disruption of hepatobiliary transport is resolved.

66. The method of claim 61 comprising conducting at least one additional clinical test of liver disease and/or liver injury.

67. The method of claim 66 wherein the at least one additional clinical test is a blood test, an imaging test, a clinical evaluation, and at a biopsy.
